(19) United States
(12) Patent Application Publication
(10) Pub. No.: US 2008/0236599 A1
(43) Pub. Date: Oct. 2, 2008

(54) Arm board adapter for surgical table

(76) Inventor: Wally Earle, Cameron (CA)

Correspondence Address: Emerson, Thomson & Bennett, LLC, 777 W. Market Street, Akron, OH, 44303, US

(21) Appl. No.: 12/077,571

(22) Filed: Mar. 20, 2008

Related U.S. Application Data
(60) Provisional application No. 60/895,833, filed on Mar. 20, 2007.

(51) Int. Cl. A61F 5/37; A61G 15/00

(52) U.S. Cl. 128/878; 128/845

(57) ABSTRACT

An adapter is disclosed. The adapter is for use with a surgical table having a bed and comprises a base portion and a pair of arm supports. The base portion overlies and is supported by the bed in use. The pair of arm supports project from opposite sides of and are supported by the base portion. The arm supports are adapted to support, in use, the forearms of a patient lying prone on said surgical table when the upper arms of said patient extend downwardly and apart from one another.

This application claims benefit of the filing date of and right of priority of U.S. Provisional Patent Application Ser. No. 60/895,833 filed Mar. 20, 2007 under 35 USC §119(e).

### FIELD OF INVENTION

The present invention relates to the field of accessories for surgical tables.

### BACKGROUND OF THE INVENTION

It is well-known for a patient to be supported on a surgical table during a surgical procedure. Specialized tables are also known, which support the use of accessories. A known accessory is an arm board, for supporting the arm of a prone patient. Existing mechanisms for accessories of this type can be relatively expensive and unwieldy. For example, it is known for such specialized tables to be provided with a rail, and for a clamp to be provided on an arm board which can be engaged to the rail when the arm board is to be used. Many practitioners and hospitals would benefit somewhat infrequently from the ability to utilize arm boards. Accordingly, many hospitals and practitioners refrain from the purchase of the arm boards and the specialized tables for receiving same. This causes the surgeon associated inconvenience when performing a procedure wherein the surgeon and/or the patient would benefit from an arm board.

### SUMMARY OF THE INVENTION

An adapter for use with a surgical table having a bed forms one aspect of the invention. The adapter comprises a base portion which overlies and is supported by the bed in use; and a pair of arm supports projecting from opposite sides of and supported by the base portion.

According to another aspect of the invention, the arm supports can be adapted to support, in use, the forearms of a patient lying prone on said surgical table when: the upper arms of said patient extend downwardly and apart from one another; and the forearms of said patient are disposed at an elevation beneath that of the bed and, when viewed in overhead plan, extends towards the head of the patient.

According to another aspect, the arm supports can be adapted to support, in use, the forearms of a patient lying prone on said surgical table when: the upper arms of said patient extend downwardly and apart from one another; and the forearms of said patient are orientated substantially horizontally and parallel to one another.

An adapter for use with a surgical table having a bed forms another aspect of the invention. This adapter comprises: a base portion adapted to overlie and which is supported by the bed in use; and a pair of wings projecting from opposite sides of and supported by the base portion.

According to another aspect, the base portion can be planar, and each wing can define a trough, each trough being orientated substantially parallel to the base portion and having a bottom surface which is disposed at an elevation beneath that of the bed in use.

According to yet another aspect, the base portion and the wings can be formed out of a single piece of sheet plastic material.

The adapters permit a conventional surgical table to be conveniently converted, when needed, into a surgical table having arm supports. Other advantages, features and characteristics of the present invention, as well as methods of operation and functions of the related elements of the structure, and the combination of parts and economies of manufacture, will become more apparent upon consideration of the following detailed description and the appended claims with reference to the accompanying drawings, the latter being briefly described hereinafter.

### BRIEF DESCRIPTION OF THE DRAWINGS

Exemplary embodiments of the invention will now be described, by way of example, with reference to the accompanying drawings, in which:

FIG. 1 is a top, right, front perspective view of an adapter constructed according to an exemplary embodiment of the invention;

FIG. 2 is a top, right, front perspective view of an adapter according to another exemplary embodiment of the invention;

FIG. 3 is a top, front overhead view of the adapter of FIG. 1 in use with a surgical table;

FIG. 4 is a top, front, left side overhead view of the structure of FIG. 1, with a cushion disposed upon the table bed and the adapter disposed thereon, and with a patient disposed in a prone position on the surgical table and in use of the adapter; and

FIG. 5 is a view of the subject-matter of FIG. 4, from the right side.

### DETAILED DESCRIPTION

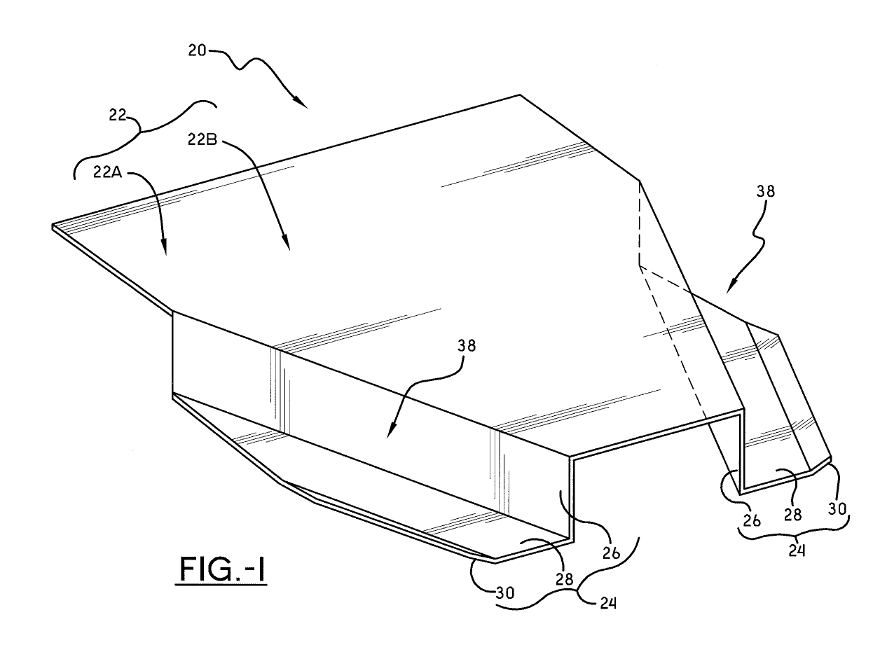

With general reference to FIG. 1, a preferred embodiment of the present invention, an adapter, is shown and designated with general reference numeral 20.

The adapter 20 has a base portion 22 and a pair of wings 24.

The base portion 22 is planar and includes a rear portion 22A and a front portion 22B. The rear portion 22A is rectangular. The front portion 22B is a truncated triangle, whose base extends from one edge of the rear portion 22A.

The wings 24 each include a flange 26, a wing body 28 and a lip 30. Flanges 26 are planar and extend one from each of the truncated sides of front portion 22B in a common direction, substantially normal or transverse to the base portion 22. The wing body 28 is substantially planar and extends, away from and substantially parallel to the base portion 22, from the flange 26. Lip portion 30 extends from the edge of the wing body 28 towards the plane of base portion 22, such that each wing 24 defines a trough 38, with the wing body 28 defining a bottom surface of the trough 38 and the troughs 38,38 extending substantially angularly or transversely to one another.

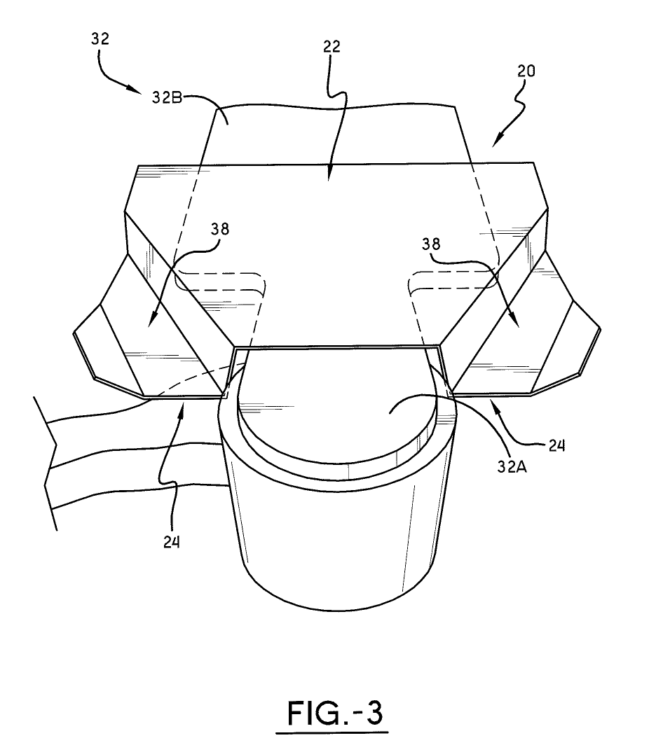

As evident from FIG. 3, wherein the adapter 20 is shown in use on a surgical table 32 with the base portion 22 overlying and supported by the bed, the base portion 22 and the wings 24 are formed (bent) out of a single rectangular piece of sheet plastic material.

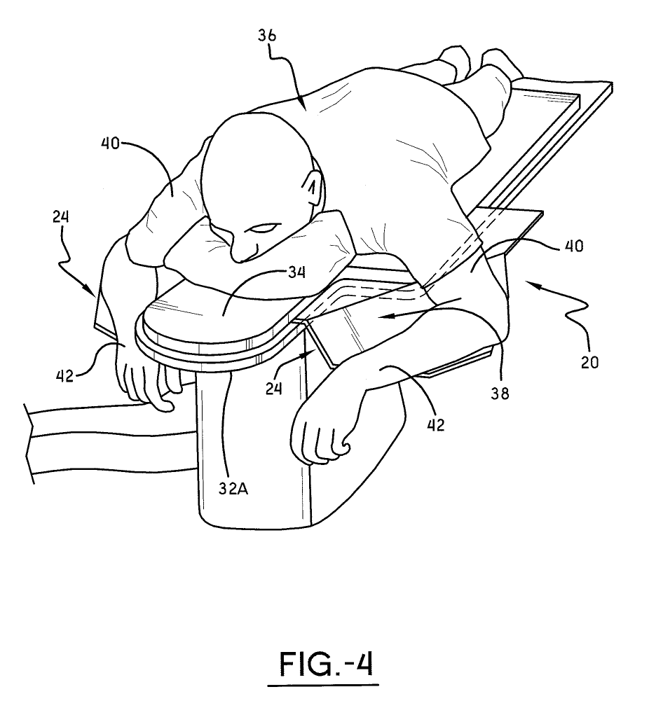

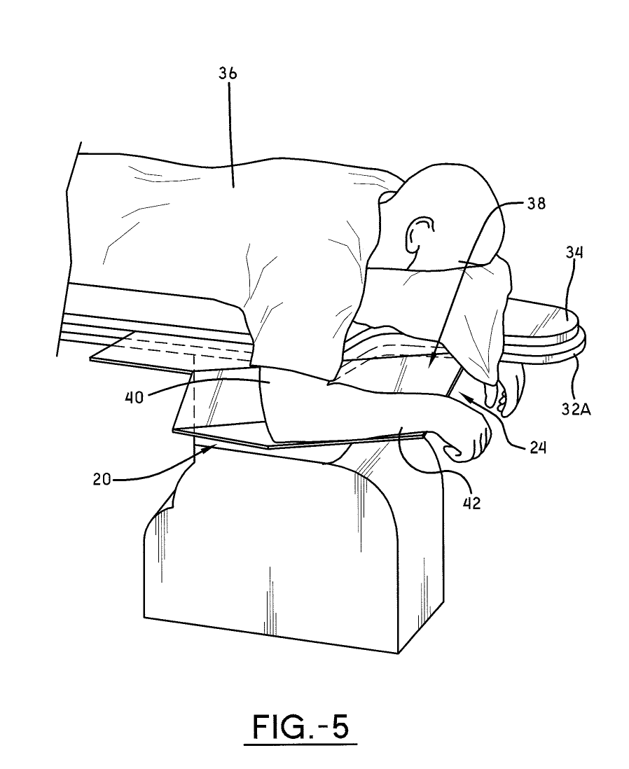

As seen in FIGS. 4, 5, wherein a cushion 34 is disposed on the adapter 20 and a patient 36 is disposed prone on the cushion 34, when the adapter 20 is in use, the wings 24 define arm supports having troughs 38 which supportingly receive the forearms 42 of a prone patient 36.

The supports 24 are configured such that the patient 36 assumes a position which is both comfortable and conducive to good blood circulation. As shown:
the upper arms 40 of said patient extend apart from one another and downwardly, i.e. the upper arms 40 of said patient are each orientated substantially parallel to a vertical plane (not shown) passing through the shoulders of said patient
the forearms 42 of said patient 36 are disposed at an elevation beneath that of the bed and orientated substantially horizontally and transversely to one another and, when viewed in overhead plan, extend towards the head of the patient.
each forearm 42 is orientated transversely to the upper arm 40 from which it extends

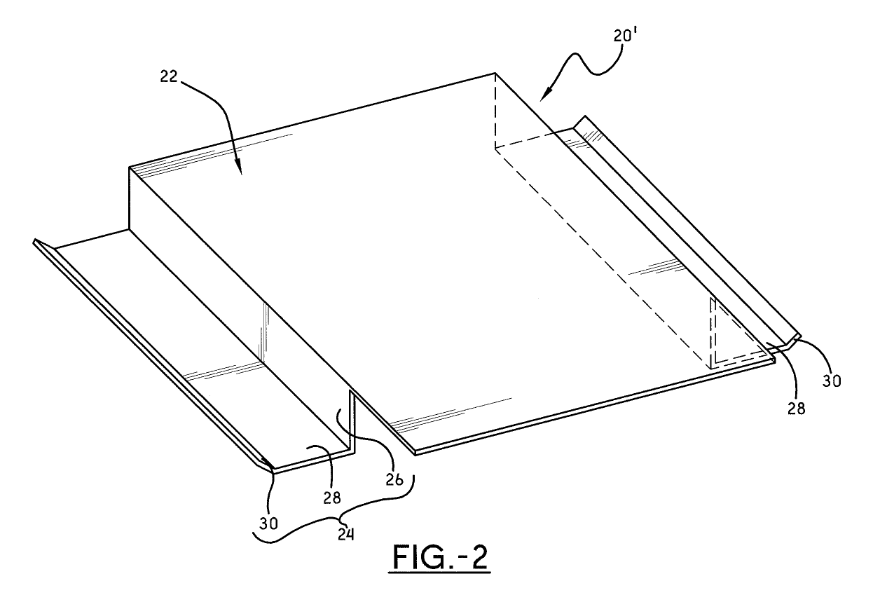

FIG. 2 shows an alternative embodiment of the adapter 20′, adapted for use with another type of conventional surgical bed (not shown), which has a rectangular bed when viewed in plan, in contrast to the bed shown in FIG. 3 which includes a head-supporting portion 32A of narrower width than the body-supporting remainder 32B of the bed. This alternative embodiment 20′ of the adapter is substantially similar in function to the adapter of FIG. 1, and is numbered identically. However, whereas the adapter of FIG. 1 supports the arms of the patient in a transverse configuration, the troughs of the adapter of FIG. 2 are orientated parallel to one another, flanking the sides of the bed in use (not shown) to support the forearms of the patient in a parallel relationship (not shown). Due to the differing geometry of the two beds, this difference in patient position has no substantial impact on comfort or circulation.

As will be readily appreciated by persons of ordinary skill, the adapter can easily be placed on a table for surgical procedures where the patient is to be prone, and removed for procedures where the patient is to be supine. The one-piece plastic construction renders the adapter easily cleaned. The relatively compact nature of the adapter permits same to be stored, for example, beneath the surgical table itself.

While but two embodiments of the present invention have been herein shown and described, it will be understood that various changes in size and shape may be made.

For example, whereas the adapter shown is formed out of a single piece of plastic material, adapters could routinely be constructed out of folded metal, or of multiple pieces of plastic or metal material secured together in any conventional fashion.

Further, whereas the troughs shown are provided with flat bottoms, other configurations, such as rounded bottoms, could be adopted.

As well, whereas in the illustrations, the troughs extend beneath the surface of the surgical table upon which the cushion rests, i.e. the bed of the surgical table, this is not necessary. For example, if a relatively thicker cushion were utilized, the lower surface of the troughs could be made level with, or even elevated with respect to the bed of the surgical table. Further, the upper surface of the adapter could be provided with a cushion.

Additionally, whereas the wings and lips of the adapter shown in FIG. 1 taper, persons of ordinary skill will readily appreciate that this is done for purposes of economy in materials usage, and other shapes could be employed.

Accordingly, it should be understood that the invention is to be limited only by the claims appended hereto, purposively construed.

1. An adapter for use with a surgical table having a bed, said adapter comprising:
a base portion which overlies and is supported by the bed in use; and
a pair of arm supports projecting from opposite sides of and supported by the base portion.

2. An adapter according to claim 1, wherein the arm supports are adapted to support, in use, the forearms of a patient lying prone on said surgical table when the upper arms of said patient extend downwardly and apart from one another.

3. An adapter according to claim 1, wherein the arm supports are adapted to support, in use, the forearms of a patient lying horizontal and prone on the table when the upper arms of said patient are each orientated substantially parallel to a vertical plane passing through the shoulders of said patient.

4. An adapter according to claim 1, wherein the arm supports are adapted to support, in use, the forearms of a patient lying prone on the table when the forearms of said patient are disposed at an elevation beneath that of the bed and orientated substantially horizontally.

5. An adapter according to claim 1, wherein the arm supports are adapted to support, in use, the forearms of a patient lying prone on the table when the forearms of said patient are orientated transversely to one another.

6. An adapter according to claim 1, wherein the arm supports are adapted to support, in use, the forearms of a patient lying prone on the table when the forearms of said patient are orientated parallel to one another.

7. An adapter according to claim 1, wherein the arm supports are adapted to support, in use, the forearms of a patient lying prone on the table when each forearm is orientated transversely to the upper arm from which it extends.

8. An adapter according to claim 1, wherein, the arm supports are adapted to support, in use, the forearms of a patient lying prone on the table when the forearms of said patient
are disposed at an elevation beneath that of the bed; and
when viewed in overhead plan, extend towards the head of the patient.

9. An adapter according to claim 1, wherein the arm supports are adapted to support, in use, the forearms of a patient lying prone on said surgical table when
the upper arms of said patient extend downwardly and apart from one another; and
the forearms of said patient
are disposed at an elevation beneath that of the bed; and
when viewed in overhead plan, extends towards the head of the patient.

10. An adapter according to claim 1, wherein the arm supports are adapted to support, in use, the forearms of a patient lying prone on said surgical table when
the upper arms of said patient extend downwardly and apart from one another; and
the forearms of said patient are orientated substantially horizontally and parallel to one another.

11. An adapter for use with a surgical table having a bed, said adapter comprising:
a base portion adapted to overlie and which is supported by the bed in use; and
a pair of wings projecting from opposite sides of and supported by the base portion.

12. An adapter according to claim 11, wherein the base portion is planar.

13. An adapter according to claim 12, wherein each wing defines a trough.

14. An adapter according to claim 13, wherein each trough is orientated substantially parallel to the base portion.

15. An adapter according to claim 12, wherein the troughs are orientated parallel to one another and flank the sides of the surgical table in use.

16. An adapter according to claim 13, wherein the troughs are orientated transversely to one another.

17. An adapted according to claim 14, wherein each trough has a bottom surface which lies at an elevation beneath that of the table top in use.

18. An adapter according to claim 11, wherein
the base portion is planar;
each wing defines a trough;
each trough is orientated substantially parallel to the base portion and has a bottom surface which lies at an elevation beneath that of the table top in use.

19. An adapter according to claim 18, wherein the base portion and the wings are formed out of a single piece of sheet plastic material.
